(19) United States
(12) Patent Application Publication
(10) Pub. No.: US 2021/0290805 A1
(43) Pub. Date: Sep. 23, 2021

(54) DEVICE AND METHOD FOR RECYCLING OF RESPIRATORY MASKS AND OTHER PERSONAL PROTECTIVE EQUIPMENT

(71) Applicant: University of Louisiana at Lafayette, Lafayette, LA (US)

(72) Inventors: Mark E. Zappi, Lafayette, LA (US); William Holmes, Lafayette, LA (US); Andrei Chistoserdov, Lafayette, LA (US); Wayne Sharp, Lafayette, LA (US); Alex Zappi, Lafayette, LA (US); Rafael Hernandez, Lafayette, LA (US); William Chirdon, Lafayette, LA (US)

(21) Appl. No.: 17/209,586

(22) Filed: Mar. 23, 2021

Related U.S. Application Data
(60) Provisional application No. 62/993,197, filed on Mar. 23, 2020.

(51) Int. Cl. A61L 2/20; A61L 2/26

(52) U.S. Cl. CPC A61L 2/202; A61F 9/02; A61L 2/26

(57) ABSTRACT

A method and device for treating contaminated or potentially contaminated items is provided. The method will include the steps of placing the items in a reactor. The reactor is then sealed and ozone or ozonated gas is sent into the reactor. The items are held in the reactor long enough to complete the decontamination process. The reactor is then opened. Next there is a period of passive reaction so that the ozone can be degraded from the pores and surfaces of the items. The device provided herein includes a reactor, an ozone generator, a rack for holding said items; and a monitor for determining the level of ozone in the reactor.

### CROSS REFERENCE TO RELATED APPLICATIONS

This application claims priority to U.S. Provisional Application No. 62/993,197, titled “Device and Method for Recycling of Respiratory Masks and Other Personal Protective Equipment” filed on Mar. 23, 2020.

### STATEMENT REGARDING FEDERALLY SPONSORED RESEARCH OR DEVELOPMENT

Not Applicable.

### REFERENCE TO A “SEQUENCE LISTING,” A TABLE, OR A COMPUTER PROGRAM

Not Applicable.

### DESCRIPTION OF THE DRAWINGS

The drawings constitute a part of this specification and include exemplary embodiments of the Device and Method for Recycling of Respiratory Masks and Other Personal Protective Equipment, which may be embodied in various forms. It is to be understood that in some instances, various aspects of the invention may be shown exaggerated or enlarged to facilitate an understanding of the invention. Therefore, the drawings may not be to scale.

FIG. 1 is a schematic depicting one embodiment of the Device and Method.

### BACKGROUND

Medical personnel, first responders, and other personnel rely on Personal Protective Equipment to protect them from infection when performing their jobs. Personal Protective Equipment (PPE) may include respiratory masks, hoods, gowns, disposable scrub sets, HazMat (Hazardous Material) suits, or other items that serve as a barrier against viruses, bacteria, fungi, or other organisms and substances that can cause illness. Adequate supplies of PPE will be critical when a particular region is faced with an epidemic or pandemic. Normally, used PPE items are thrown out as waste and cannot be reused. While normal levels of PPE supplies may initially be adequate in the face of an outbreak of communicable diseases, as the pandemic spreads those supplies will likely be exhausted.

The invention described herein provides a device and method for treating used PPE items so that they can be used again. While the description and claims described herein focus on PPE items, those skilled in the art will find other applications for the device and method. For example, the invention may be applied for treatment of bed linens, surgical items, or any other materials that may have been exposed to virus particles or other pathogens.

### DETAILED DESCRIPTION

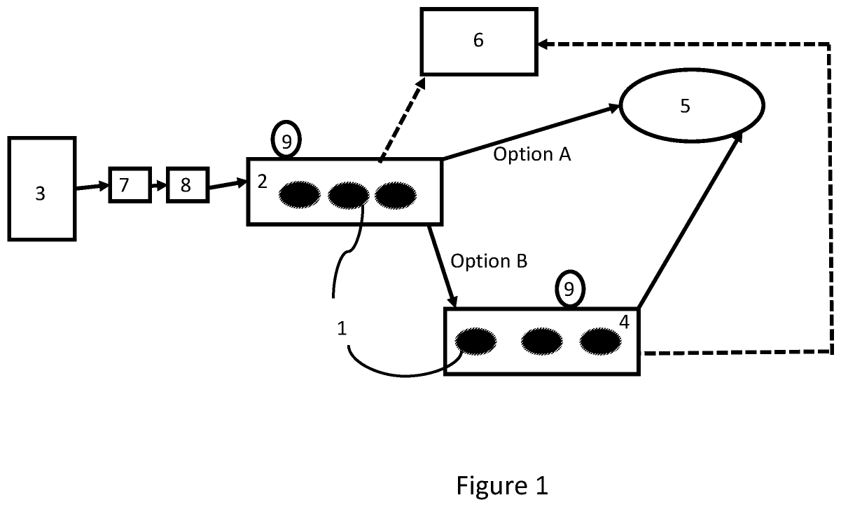

This device will utilize ozone gas (O₃) as the active agent for the disinfection method. With reference to FIG. 1, contaminated or potentially contaminated PPE items 1 are placed within reactor 2. PPE items 1 may include respirators (ex. N95), protective suits, goggles, and other personal PPE items. After PPE items 1 are placed within reactor 2, reactor 2 is sealed and ozonated air and/or ozonated oxygen is gassed into the sealed reactor. The ozonated gas is kept in the sealed reactor long enough so that ozonated gas has sufficient time to interact completely with the potentially contaminated surfaces of the objects to be disinfected. For most disinfection applications the surfaces of typical PPE items will require only seconds to minutes of treatment in the reactor.

The ozonated gas is generated on-site using a portable or fixed ozone generator 3 (commercial or lab grade) using air and/or oxygen as the oxygen source. One type of ozone generator known to those skilled in the art is the Corona Discharge ozone generator. However, any other type of ozone generator known to those skilled in the art will suffice. Ozone generator 3 will be plumbed so that the ozonated gas is forced into the reactor containing the potentially contaminated/infected PPE. In one embodiment, ozonated gas will be pass through flowmeter 7 and mass totalizer 8 that are place intermediate ozone generator 3 and reactor 2.

After sufficient time is allowed for the ozone to disinfect the pathogen; under Option B the gas may be released into secondary reactor 4 that can serve as a pretreatment step, thus saving ozone. In an alternative embodiment (Option A), the used ozonated gas is passed to ozone destruction unit 5 to purge the carrier gas of any ozone (ozone is considered a pollutant).

Once the gas pressure within the primary reactor 2 is equalized to atmospheric pressure, then a period of passive reaction can be performed to allow the PPE to degrade the ozone from within its pores and/or surfaces. Because ozone is not a stable gas it will likely have to be generated on-site. The unstable character of ozonated gas is important to the method because this characteristic will allow passive removal of the ozone from the PPE. Pressures in reactor 2 and secondary reactor 4 will be monitored using pressure gauges 9.

This process can be utilized at a small, single PPE scale (e.g. a single N95 mask) or scaled to very large application (several HazMat suits). Additionally, the device and method may be used for decontamination of medical devices, medical instruments, lab equipment, emergency equipment, or any other item needed decontamination.

The ozone gas contact and treatment device and method described herein disinfects the PPE items of pathogens and other hazards, including but not limited to viruses (including the corona virus or COVID 19 virus), bacteria, and fungi.

In a preferred embodiment, reactor 2 comprises a tubular reaction/disinfection reactor. In a particularly preferred embodiment, the ozonated gas ozone concentrations are continuously monitored using an ozone monitoring system. In one embodiment the ozone monitoring system will comprise real-time gas ozone gas analyzer 6 with sample points at ozone generator 3 and along the length of the reactor tube to ensure that appropriate levels of ozone are present. The device can be configured so that ozonated gas can be introduced into either end of reactor 2 or run only from one direction.

Multiple secondary reactors 4 can be plumbed in series to fully use the ozone already generated so that as ozonated gas exits the primary disinfection reactor, that gas can be passed through a secondary or tertiary reactor system which fully uses the generated ozone.

In one embodiment, the disinfection reactors can also be fully charged with ozonated gas and run as true batch systems such that the reactor is fully charged with ozonated gas and the reactions allowed to occur until some C-T dose (concentration-contact time=C-T) is reached. In another embodiment, a wire rack is used to hold the masks upright and the cupped part of a concave mask is facing the incoming gas stream to maximize gas contact with the pores of the materials being treated. In another embodiment, a UV lamp can be inserted into the reactor to provide secondary disinfection in conjunction with ozone, thus producing hydroxyl radicals.

In another embodiment, The PPE items laid out on the reactor support rack can be removed easily from the reactor, then inserted into reactor 2 with only compressed air and/or nitrogen and/or carbon dioxide and/or any other gas to assist in the removal of the residual ozone from the PPE. In a further embodiment, UV lamps can be inserted into the de-ozonation reactor to assist and accelerate the dissipation of ozone residuals. The residual ozone within the ozonated gas may be removed from the final exiting gas stream using a commercial ozone destruction system.

In another embodiment, the primary and secondary reactors can be filled with ozonated water to use a wet disinfection mode within the reactors. However, for many PPE items (e.g. N95 masks), wet disinfection will not be suitable. In another embodiment, hydrogen peroxide can be batch added or continuously added along with ozonation to initiate peroxone reactions to produce large amounts of the hydroxyl radical, which is a much more effective disinfection agent than ozone alone. Hydrogen peroxide can also be added without ozone but with the addition of iron salts to use Fenton's Reaction within the reactors to produce the hydroxyl radical.

In another embodiment, the reactor can be configured so that it resembles a sealed hanging clothes closet/box to allow for disinfection of hanging PPE. The rack can be modified to hold open gloves set into the reactor for disinfection. It will be obvious to those skilled in the art to disinfect other medical devices using both reactor designs—those using ozone alone those using hydrogen peroxide and UV light alone or in combination with ozone.

In another embodiment, in hydrogen peroxide embodiments, the solution pH in the reactor may be increased to allow for ozone to be converted into the hydroxyl radical. The disinfection system can be fully automated using controls and solenoids. An ozone monitor can be installed onto the exit ports of the deozonation reactor to ensure no residual ozone is within the exit gas.

In another embodiment, the temperature of disinfection and deozonation within the reactors can be elevated to enhance the rate of reaction. This can be accomplished using a variety of heating options, including but not limited to heat wrapping, annular tubed reactor, pre-reactor heat exchanger, and other options known to those skilled in the art. Baffles can be added into both the disinfection and deozonation reactors to increase the mixing of the gases within the reactors.

Vacuum may be applied prior to the injection of either the ozonated gas for the disinfection reactor or the sweep gas within the deozonation reactor to enhance the gas entrance into the small pores of the PPE. A variety of reactor configurations may be used, such as a box or some other more dimensionally equal reactor, not just tube reactors.

An in-line section of vortexing fins can be added into the reactors. The ozonated gas and/or liquids can be pulsed and/or oscillated within the reactors to improve pore access of the disinfection agents. Ultrasound can be added to the reactors to enhance mass transfer and completion of the reactions.

In another embodiment, ozonated gas may be directed injected into the PPE within an encased vessel using a directed gas spray—similar to a sand blasting box or glove box.

A pressure-drop in-line vortex or venture tube gas introduction system can be used to introduce liquid reactants into the reactors. Mass flow totalizers and pressure gauges can be added before, along, and after the reactors to monitor process conditions. Fans or some similar mixing and velocity accelerators may added to the reactors to enhance mass transfer. The ozonated gas and/or the deozonation gas does not have to be introduced at the ends of the reactor—the gas can be introduced anywhere along the length of the reactors or any combination of several injection points on the reactors.

The disinfection nature of the proposed invention is not only limited to ozone but also oxidizer radicals, hydrogen peroxide, UV photons, heat, and/or any other disinfection mechanism(s).

The subject matter of the present invention is described with specificity herein to meet statutory requirements. However, the description itself is not intended to necessarily limit the scope of claims. Rather, the claimed subject matter might be embodied in other ways to include different steps or combinations of steps similar to the ones described in this document, in conjunction with other present or future technologies.

1. A method of treating contaminated or potentially contaminated items, comprising:
a. Providing a reactor;
b. Placing said items in said reactor;
c. Sealing said reactor;
d. Gassing ozone gas or ozonated gas into said reactor;
e. Keeping said items in reactor long enough so that ozone gas or ozonated gas has sufficient time to interact completely with the potentially contaminated surfaces of said items;
f. Unsealing said reactor such that gas pressure within said reactor is equalized to atmospheric pressure; and
g. Allowing a period of passive reaction so that said items can degrade the ozone from within their pores and surfaces.

2. The method of claim 1, further comprising, after unsealing the reactor, releasing said ozone gas or ozonated gas into a secondary reactor that can be used for pretreatment of said items.

3. A device for treating contaminated or potentially contaminated items, comprising:
a. A reactor having an opening;
b. An ozone generator connected to said reactor;
c. A rack for holding said items;
d. A monitor for determining the level of said ozone in said reactor.
